SEQUENCE LISTING (16 sequences)

SEQ ID NO: 1 - DNA, 83 bp, Artificial
  Primer 1
agtgaattgtaatacgactcactatagggcgaattcgagctcggtacccgcttaagtacggctacctggagctcgcgccagtg

SEQ ID NO: 2 - DNA, 114 bp, artificial
  Primer 2
tacggctacctggagctcgcgccagtgcttgccgacatcgaatcacggccgctgctgcagcacgtggtggtcaccggccgggatccgtttaaacacaaatggcaagggctaatg

SEQ ID NO: 3 - DNA, 114 bp, artificial
  Primer 3
attgatgttttccttggccagcgcctcgaacatccggctggcgacgcctgcgtgcgaacgcataccgacaccgacgatagggatccgtttaaacggtgtggtagctcgcgtatt

SEQ ID NO: 4 - DNA, 85 bp, artificial
  Primer 4
gcgacactatagaatactcaagcttggcatgaatgcaggtcgactctagagaatattgatgttttccttggccagcgcctcgaac

SEQ ID NO: 5 - DNA, 18 bp, artificial
  NiF-check-1
ggtctaccagctcgacct

SEQ ID NO: 6 - DNA, 20 bp, artificial
  NiF-check-2
cgattccagcgtcgaatgat

SEQ ID NO: 7 - DNA, 20 bp, artificial
  NiF-check-3
gctgacctccttgaggtgct

SEQ ID NO: 8 - DNA, 19 bp, artificial
  NiF-check-4
cagcggcacctcgaggagt

SEQ ID NO: 9 - DNA, 19 bp, artificial
  NiF-check-5
gatagagcaggtcctcgat

SEQ ID NO: 10 - DNA, 20 bp, artificial
  NiF-check-6
ggtgctctacgtcagccatt

SEQ ID NO: 11 - DNA, 20 bp, artificial
  NiF-check-7
cgacagatcctgattaccgt

SEQ ID NO: 12 - DNA, 21 bp, artificial
  NiF-check-8
taccctcgaccagcttgagca

SEQ ID NO: 13 - DNA, 21 bp, artificial
  check-5′
tgcttctaccgcaaggacatc

SEQ ID NO: 14 - DNA, 21 bp, artificial
  check-3′
gctgatgaagcacgagagcac

SEQ ID NO: 15 - DNA, 97 bp, artificial
  rets-genta-loxm-5′
gcacacgcccttgccgtgcggtcattacgccgcgcatagttataatcaggcatcaaccaacgaagggatttcgccagctgaattacattcccaaccg

SEQ ID NO: 16 - DNA, 98 bp, artificial
  rets-genta-loxm-3′
tggagcatggtgggagctcacgactaaaggagggcgagcgagagtttaacaggcgccgcagagcctgtcggctcacaacttaaatgtgaaagtgggtc

(19) United States
(12) Patent Application Publication
(10) Pub. No.: US 2020/0120939 A1
(43) Pub. Date: Apr. 23, 2020

(54) Method For Screening Pseudomonas Protegens Mutant Strain, And Application Thereof In Biological Control

(71) Applicants: Shandong University, Jinan City, Shandong (CN); Dezhou Microp Bio-Technology Co., Ltd., Yucheng City, Shandong (CN)

(72) Inventors: Youming Zhang, Jinan City, Shandong (CN); Qiang Tu, Jinan City (CN); Fangnan Yu, Yucheng City, Shandong (CN); Xiaoshu Jing, Jinan City, Shandong (CN); Xiaoying Bian, Jinan City, Shandong (CN); Hanna Chen, Jinan City, Shandong (CN)

(21) Appl. No.: 16/603,938

(22) PCT Filed: Apr. 18, 2018
(86) PCT No.: PCT/CN2018/083457

§ 371 Date: Oct. 9, 2019

(30) Foreign Application Priority Data
Apr. 19, 2017 (CN) 201710256234.6

(51) Int. Cl. A01N 63/27; C07K 14/21

(52) U.S. Cl. CPC A01N 63/27; C07K 14/21

(57) ABSTRACT

Provided are Pseudomonas protegens mutant strain Pf5-NiF, Pf5-ΔretS, or Pf5-ΔretS-NiF, and a screening method therefor and an application thereof. By means of Red/ET recombination and direct cloning technologies, the NiF nitrogen fixation gene island in the genome of Pseudomonas stutzeri DSM4166, taken as a whole, is cloned into the genome of Pseudomonas protegens Pf5, so as to heterologously express the same successfully to obtain a genetically engineered strain Pf5-NiF, thereby bringing a biological nitrogen fixation function to Pseudomonas protegens Pf5 which does not own a biological nitrogen fixation function. In addition, gene-directed markerless knockout of retS gene in the genome of Pseudomonas protegens Pf5 is performed to obtain a genetically engineered strain Pf5-ΔretS. Thus, the expression levels of an antibiotic 2,4-diacetylphloroglucinol and red pigment are increased, and a mutant strain of Pseudomonas protegens Pf5 having a stronger bactericidal activity is obtained.

### FIELD OF THE INVENTION

The invention belongs to the field of biotechnology. In particular, the present invention relates to a mutant strain of Pseudomonas protegens, and more particularly to a method for screening a mutant strain of Pseudomonas protegens and its use in biological control.

### BACKGROUND

Pseudomonas protegens is a Grain-negative rod-shaped bacterium commonly found in plant rhizosphere and soils. It is one of the most studied bacteria among plant growth-promoting rhizobacteria (PGPR). Because of its rapid reproduction speed, strong adaptability, easy artificial cultivation, stable formulation, convenient administration, no pollution to the environment, prevention and treatment of various plant diseases, etc., the Chinese Ministry of Agriculture has listed it as one of the registrable microbial pesticides and fertilizer varieties, and is promoted and used nationwide. Because it can produce one or more antibiotics, such as 2,4-diacetylphloroglucinol (2,4-DAPG), pylonuteorin (Plt), Phenazine, pyrrolnitrin (Pm), AprA protease and hydrocyanic acid (HCN), etc., it has good preventive effects for plant diseases, especially soil-borne diseases such as squatting, root rot, blight, etc., and is an important class of beneficial microbial resources, which is of great significance in agricultural production. However, Pseudomonas protegens itself does not have the function of biological nitrogen fixation.

Pseudomonas stutzeri DSM4166 is a joint nitrogen-fixing bacteria isolated from the rhizosphere soil of German sorghum. It has been deposited in the German Collection of Microorganisms (Deutsche SammLung von Mikroorganismen and Zellkulturen GmbH) and assigned a deposit number DSM4166. This bacterium can convert nitrogen in the air into ammonium which can be directly utilized by plants under ammonia-free and micro-aerobic conditions. At present, the genome sequencing work of this strain has been completed, and a 69 kb NiF nitrogen-fixing gene island has been found in the genome sequence, which contains 58 different genes. However, the results of genome sequencing indicated that the secondary metabolites of the bacterium were not abundant and the antibacterial ability was weak.

Red/ET homologous recombination and direct cloning technology is a novel genetic engineering technology based on phage recombinase. Its basic principle is to modify the DNA sequence by phage recombinase-mediated homologous recombination in E. coli. This technology is not limited by the size and restriction sites of DNA molecules, and can accurately and efficiently perform gene insertion, gene knockout, point mutation and module replacement on gene clusters. Higher recombination efficiency can be obtained by only using 30-50 bp size homologous arms. The direct cloning technology uses the RecET recombinase to clone the natural product gene cluster directly from the microbial genome into the E. coli expression vector, and the entire gene cluster can be cloned in only 3 days, and the recombination step occurs in the E. coli cells to avoid the mutation of the gene clusters during cloning. At present, this technology has been widely used in the cloning and heterologous expression studies of microbial natural product gene clusters.

### SUMMARY OF THE INVENTION

It is an object of the present invention to provide Pseudomonas protegens having both the disease prevention and growth promoting effects in order to overcome the deficiencies of the prior art described above.

The invention also provides a method for screening the above strain and the use thereof in biological control.

In order to achieve the above object, the present invention adopts the following technical solutions:

Pseudomonas protegens Pf5 mutant strain Pf5-NiF, Pf5-ΔretS or Pf5-ΔretS-NiF, having the deposit numbers CGMCC NO. 13948, CGMCC NO. 13949 and CGMCC NO. 13950 respectively (deposited in China General Microbiological Culture Collection Center Institute of Microbiology, Chinese Academy of Sciences, NO. 1 West Beichen Road, Chaoyang District, Beijing 100101, China on Mar. 28, 2017).

Use of Pseudomonas protegens Pf5 mutant strain Pf5-NiF in promoting plant growth, killing bacteria and fixing nitrogen.

Use of Pseudomonas protegens Pf5 mutant strain Pf5-ΔretS in promoting plant growth and killing bacteria.

Use of Pseudomonas protegens Pf5 mutant strain Pf5-ΔretS-NiF in promoting plant growth, killing bacteria and fixing nitrogen.

A composition, for example, a microbial agent, which has Pf5-NiF or Pf5-ΔretS or Pf5-ΔretS-NiF, or any combination thereof as the active ingredient.

A method for screening Pseudomonas protegens Pf5 mutant strain Pf5-NiF, comprising cloning the whole NiF nitrogen-fixing gene island in the genome of Pseudomonas stutzeri DSM4166 into the genome of Pseudomonas protegens Pf5, and expressing the NiF nitrogen-fixing gene island to obtain the genetically engineered strain Pf5-NiF.

As one of the preferred technical solutions, the specific steps are as follows:

(1) Using Red/ET direct cloning method, the restriction endonucleases Afl II and Ssp I were used to digest the genomic DNA of Pseudomonas stutzeri DSM4166 to obtain a 69 kb NiF nitrogen-fixing gene island, which was ligated to the corresponding vector to construct the plasmid. The plasmid pBeloBAC11-oriT-TnpA-genta-NiF was constructed using the primers as shown in SEQ ID NO. 1, SEQ ID NO. 2, SEQ ID NO. 3 and SEQ ID NO. 4, and was identified by digesting with restriction endonuclease Kpn I. The correct plasmid was electrotransformed into E. coli ET12567;

(2) The plasmid pBeloBAC11-oriT-TnpA-genta-NiF from E. coli ET12567 was introduced into Pseudomonas protegens Pf5 by conjugative transfer, and then NiF gene was randomly inserted into the genomic DNA of Pf5 by transposition;

(3) The correct transformant Pf5-NiF was sent to the sequencing after colony PCR verification, and that with the correct results was cryopreserved.

As one of the further preferred technical solutions, the specific process of step (2) is: A single colony of Pseudomonas protegens Pf5 was picked up and was cultured (LB medium (tryptone 10 g/L, yeast extract 5 g/L, NaCl 5 g/L, pH adjusted to 7.0), 30° C.) separately with E. coli ET12567 (LB+genta 2 μg/mL+cm 10 μg/mL+kin 1 μg/mL medium, 37° C.) overnight; The two overnight bacterial solutions were centrifuged at 7000 rpm for 1 minute. Pseudomonas protegens Pf5 and E. coli ET12567 were washed twice with fresh LB medium, resuspended in 300 μL of LB medium. 50 μL of each suspension was mixed and coated on a small area in the middle of the LB plate and air dried. After incubating for 4 hours at 37° C., the plate was invertedly incubated in an incubator at 30° C. overnight; The bacteria on the plate were scraped with an inoculating loop, suspended with sterilized solution and mixed. 100 μL of bacterial solution was streaked in a Z-shaped line on PMM (K₂HPO₄ 5 g/L, KH₂PO₄ 5 g/L, ammonium sulfate 1 g/L, sodium succinate 6.6 g/L, adjusted to pH 7.0, 1M magnesium sulfate 1.2 mL/L was added after steralization)+genta 25 μg/mL+Amp 100 μg/mL solid medium, and cultured invertedly at 30° C. for 2 days until single colonies appeared; A single colony was picked up to be inoculated in 1 mL of LB+genta 25 μg/mL+Amp 100 μg/mL and to be cultured overnight, followed by colony PCR verification using the 5 pairs of primers shown in SEQ ID NO. 5 to SEQ ID NO. 14.

A method for screening Pseudomonas protegens Pf5 mutant strain Pf5-ΔretS, comprising scarlessly knocking out retS gene from the genome of Pseudomonas protegens Pf5 to obtain the genetically engineered strain Pf5-ΔretS.

As one of the preferred technical solutions, the specific steps are as follows:

(1) The plasmid pBBR1-Rha-TEGpsy-kan was introduced into the wild type Pseudomonas protegens Pf5 by electrotransformation, and the correct transformant Pf5::pBBR1-Rha-TEGpsy-kan was screened;

(2) retS gene in the genome of Pseudomonas protegens Pf5 was knocked out;

(3) The correct transformant Pf5-ΔretS was cryopreserved after PCR verification and sequencing.

As one of the further preferred technical solutions, the specific process of step (1) is: The electrotransformed bacteria were coated on a plate of LB medium containing 30 μg/mL kanamycin, and single colonies were selected randomly to extract the plasmids to be enzymatically identified, and the correct transformant Pf5::pBBR1-Rha-TEGpsy-kan was screened.

As one of the further preferred technical solutions, the specific process of step (2) is:

(21) The linear DNA fragment loxM-genta was electrotransformed into Pf5::pBBR1-Rha-TEGpsy-kan obtained in step (1). Using the method of Red/ET homologous recombination, under the action of recombinase, retS gene in the genome of Pseudomonas protegens Pf5 was replaced by gentamicin resistance gene genta, and the correct transformant Pf5::ΔretS-genta-loxM was screened by culture.

(22) The PCM157 plasmid capable of expressing Cre recombinase was electrotransformed into Pf5::ΔretS-genta-loxM, cultured and screened, induced by isopropyl-β-D-thiogalactoside (IPTG), and recombinants whose genta resistance gene has been eliminated were picked up to be subjected to PCR verification and sequencing.

As one of the further preferred embodiments, the linear DNA fragment loxM-genta in step (21) was obtained by PCR amplification using a pair of primers shown in SEQ ID NO. 15 and SEQ ID NO. 16.

As one of the further preferred embodiments, the culture screening method of step (21) was: The recombinant bacteria were coated on a plate of LB medium containing 15 μg/mL kanamycin, and single colonies were selected randomly for colony PCR verification, and the correct transformant Pf5::ΔretS-genta-loxM was screened.

As one of the further preferred embodiments, PCR verification was carried out by PCR amplification using a pair of primers shown in SEQ ID NO. 13 and SEQ ID NO. 14.

As one of the further preferred embodiments, the specific process of the step (22) is: PCM157 plasmid capable of expressing Cre recombinase was electrotransformed into Pf5::ΔretS-genta-loxM, and was coated on a plate of LB medium containing 25 μg/mL tetracycline for screening; The resultant recombinants were inoculated into 1 mL of LB medium containing 25 μg/mL tetracycline, and cultured at 900 rpm, 30° C. overnight; The next day, 50 μL of the overnight cultured bacterial solution was transferred to 1 mL of fresh LB medium containing 25 μg/mL of tetracycline; After culturing at 900 rpm for 3 hours at 30° C., 1 mM of isopropyl-β-D-thiogalactoside (IPTG) was added for induction. After continuing the culture for 2 hours, the bacterial solution was streaked in a Z-shaped line on a LB plate and a plate of LB medium containing 15 μg/mL gentamycin respectively with a blue inoculation loop and cultured at 30° C. overnight. If single colonies grew on both plates, it indicated that the genta resistance gene in the recombinant had not been eliminated; If the colonies grew on LB plate while did not grow on the LB+genta 15 μg/m plate, it indicated that the genta resistance gene in the recombinant had been eliminated; Such recombinants whose genta resistance gene had been eliminated were picked up and subjected to colony PCR verification and sequencing.

As one of the further preferred embodiments, PCR verification was carried out by PCR amplification using a pair of primers shown in SEQ ID NO. 13 and SEQ ID NO. 14.

A method for screening Pseudomonas protegens Pf5 mutant strain Pf5-ΔretS-NiF, comprising introducing the plasmid pBeloBAC11-oriT-TnpA-genta-NiF from E. coli ET12567 into Pseudomonas protegens Pf5-ΔretS by conjugative transfer, and then randomly inserting NiF gene into the genomic DNA of Pf5-ΔretS by transposition.

As one of the preferred technical solutions, the specific steps are as follows:

(1) The plasmid pBBR1-Rha-TEGpsy-kan was introduced into the wild type Pseudomonas protegens Pf5 by electrotransformation, and the correct transformant Pf5::pBBR1-Rha-TEGpsy-kan was screened;

(2) retS gene in the genome of Pseudomonas protegens Pf5 was knocked out to obtain the mutant Pseudomonas protegens Pf5-ΔretS;

(3) Using Red/ET direct cloning method, the restriction endonucleases Afl II and Ssp I were used to digest the genomic DNA of Pseudomonas stutzeri DSM4166 to obtain a 69 kb NiF nitrogen-fixing gene island, which was ligated to the corresponding vector after verification by DNA fragment electrophoresis. The plasmid pBeloBAC11-oriT-TnpA-genta-NiF was constructed using the primers as shown in SEQ ID NO. 1, SEQ ID NO. 2, SEQ ID NO. 3 and SEQ ID NO. 4, and was identified by digesting with restriction endonuclease Kpn I. The correct plasmid was electrotransformed into E. coli ET12567;

(4) The plasmid pBeloBAC11-oriT-TnpA-genta-NiF from E. coli ET12567 was introduced into the mutant Pseudomonas protegens Pf5-ΔretS by conjugative transfer. Then, NiF gene was randomly inserted into the genomic DNA of Pf5-ΔretS by transposition.

As one of the further preferred technical solutions, the specific process of step (1) is: The electrotransformed bacteria were coated on a plate of LB medium containing 30 μg/mL kanamycin, and single colonies were selected randomly to extract the plasmids to be enzymatically identified, and the correct transformant Pf5::pBBR1-Rha-TEGpsy-kan was screened.

As one of the further preferred technical solutions, the specific process of step (2) is:

(21) The linear DNA fragment loxM-genta was electrotransformed into Pf5::pBBR1-Rha-TEGpsy-kan obtained in step (1). Using the method of Red/ET homologous recombination, under the action of recombinase, retS gene in the genome of Pseudomonas protegees Pf5 was replaced by gentamicin resistance gene genta, and the correct transformant Pf5::ΔretS-genta-loxM was screened by culture.

(22) The PCM157 plasmid capable of expressing Cre recombinase was electrotransformed into Pf5::ΔretS-genta-loxM, cultured and screened, induced by isopropyl-β-D-thiogalactoside (IPTG), and recombinants whose genta resistance gene has been eliminated were picked up to be subjected to PCR verification and sequencing.

As one of the further preferred embodiments, the linear DNA fragment loxM-genta in step (21) was obtained by PCR amplification using a pair of primers shown in SEQ ID NO. 15 and SEQ ID NO. 16.

As one of the further preferred embodiments, the culture screening method of step (21) was: The recombinant bacteria were coated on a plate of LB medium containing 15 μg/mL kanamycin, and single colonies were selected randomly for colony PCR verification, and the correct transformant Pf5::ΔretS-genta-loxM was screened.

As one of the further preferred embodiments, PCR verification was carried out by PCR amplification using a pair of primers shown in SEQ ID NO. 13 and SEQ ID NO. 14.

As one of the further preferred embodiments, the specific process of the step (22) is: PCM157 plasmid capable of expressing Cre recombinase was electrotransformed into Pf5::ΔretS-genta-loxM, and was coated on a plate of LB medium containing 25 μg/mL tetracycline for screening; The resultant recombinants were inoculated into 1 mL of LB medium containing 25 μg/mL tetracycline, and cultured at 900 rpm, 30° C. overnight; The next day, 50 μL of the overnight cultured bacterial solution was transferred to 1 mL of fresh LB medium containing 25 μg/mL of tetracycline; After culturing at 900 rpm for 3 hours at 30° C., 1 mM of isopropyl-β-D-thiogalactoside (IPTG) was added for induction. After continuing the culture for 2 hours, the bacterial solution was streaked in a Z-shaped line on a LB plate and a plate of LB medium containing 15 μg/mL gentamycin respectively with a blue inoculation loop and cultured at 30° C. overnight. If single colonies grew on both plates, it indicated that the genta resistance gene in the recombinant had not been eliminated; If the colonies grew on LB plate while did not grow on the LB+genta 15 μg/m plate, it indicated that the genta resistance gene in the recombinant had been eliminated; Such recombinants whose genta resistance gene had been eliminated were picked up and subjected to colony PCR verification and sequencing.

As one of the further preferred embodiments, PCR verification was carried out by PCR amplification using a pair of primers shown in SEQ ID NO. 13 and SEQ ID NO. 14.

As one of the further preferred technical solutions, the specific process of the step (4) is: The mutant Pseudomonas protegens Pf5-ΔretS (LB medium, 30° C.) and E. coli ET12567 (LB+genta 2 μg/mL medium, 37° C.) were cultured overnight respectively; The next day, the mutant Pseudomonas protegens Pf5-ΔretS and E. coli ET12567 were washed twice with fresh LB medium respectively, and dissolved in 300 μL of LB in the same amounts respectively, and then mixed together to 600 μL. After centrifuged at 9000 rpm for 1 minute, most of the supernatant was discarded. 50 μL of the bacterial solution was retained to be resuspended with the mixed bacteria, and uniformly coated on a LB plate in a small range. After incubated at 37° C. for 4 hours, the plate was placed in an incubator at 30° C. to be cultured overnight; On the third day, the co-cultured bacteria were transferred with a yellow inoculating loop from the LB plate to 1 mL of LB to be mixed thoroughly. 30 μL of the bacterial solution was streaked in a Z-shaped line on PMM medium+genta 25 μg/mL+Amp 100 μg/mL. Two days later, colonies were grown. Single colonies were picked up and inoculated in 1 mL LB+genta 25 μg/mL+Amp 100 μg/mL to be cultured overnight, and then subjected to colony PCR verification using the 5 pairs of primers shown in SEQ ID NO. 5 to SEQ ID NO. 14. The correct transformant Pf5-ΔretS-NiF was sent to the sequencing after PCR verification, and that with the correct results was cryopreserved.

The present invention further relates to a method for promoting plant growth, killing bacteria and/or fixing nitrogen, comprising administering to a plant or a seed thereof Pseudomonas protegens mutant strain Pf5-NiF or Pf5-ΔretS-NiF or a combination thereof, or a composition, for example, a microbial agent, comprising Pseudomonas protegens mutant strain Pf5-NiF or Pf5-ΔretS-NiF or a combination thereof.

The present invention also relates to a method for promoting plant growth and/or killing bacteria, comprising administering to a plant or a seed thereof Pseudomonas protegens mutant strain Pf5-ΔretS, or a composition, for example, a microbial agent, comprising Pseudomonas protegens mutant strain Pf5-ΔretS.

The plants to which the present invention relates may be monocotyledonous or dicotyledonous plants, such as plants of cruciferae, Gramineae, liliaceae, and the like.

The beneficial effects of the invention:

The invention utilizes Red/ET recombination and direct cloning technologies to clone the NiF nitrogen-fixing gene island in the genome of Pseudomonas stutzeri DSM4166 into the genome of Pseudomonas protegens Pf5 to make it successfully expressed to obtain the genetically engineered strain Pf5-NiF. Thus, Pseudomonas protegens Pf5, which otherwise has no biological nitrogen fixation, can produce biological nitrogen fixation function. In addition, retS gene in the genome of Pseudomonas protegens Pf5 was scarlessly knocked out to obtained the genetically engineered strain Pf5-ΔretS to increase the expression level of the antibiotic 2,4-diacetylphloroglucinol (2,4-DAPG) and red pigment, so that a mutant strain of Pseudomonas protegens Pf5 having stronger bactericidal activity was obtained. Pseudomonas protegens Pf5 has never been reported to be capable of fixing nitrogen by itself or used for plant growth promoting properties. After the genetically engineered strain Pf5-NiF was applied to different crops cultivated under greenhouse and field conditions, it provided significant biological nitrogen fixation and growth promoting effects. Moreover, according to available data, its action has consistently been more stable and reproducible than any other previously recorded plant growth promoting microbial preparations.

The invention provides Pseudomonas protegens mutant strain Pf5-ΔretS and a bacterial agent comprising it as an active ingredient. Potting at room temperature and field trials prove that the strain has the dual functions of preventing diseases and promoting growth. It not only has good control effects for soil-borne diseases, such rickets, root rot, blight, etc. of various plants, but also can effectively promote growth of plants. Genomics and molecular biology studies have shown that the main mechanism of this strain to control plant diseases is its ability to produce antibiotics that inhibit the growth of pathogenic bacteria, such as 2,4-diacetylphloroglucinol (2,4-DAPG) and pyoluteorinetc, as well as good plant rhizosphere colonization ability. The main mechanism for promoting plant growth is that it contains 1-aminocyclopropane-1-carboxylate deaminase gene. The enzyme reduces the ethylene content in and around the roots of the plant seedlings, thereby stimulating plant growth.

Pseudomonas protegens Pf5 and its mutant strains Pf5-NiF, Pf5-ΔretS and Pf5-ΔretS-NiF can grow single colonies after 2-3 days of culture on LB solid medium at 30° C. Single colonies were picked up and inoculated into LB liquid medium (appropriate antibiotics can be added for culture and screening), followed by subsequent culture and genetic manipulation, and the transformants of corresponding genetically engineered strains were obtained. After enzymatically identification and sequencing, the correct genetically engineered bacteria were cultured in large scale for potting at room temperature and field trials.

The present invention provides a bacterial agent prepared from Pseudomonas protegens Pf5 and its mutant strains Pf5-NiF, Pf5-ΔretS and Pf5-ΔretS-NiF, which comprises no less than 1×10⁹ cfu/mL of Pseudomonas protegens Pf5. The bacterial agent has simple preparation process and short fermentation cycle, and has great potential for industrialized production. The invention has wide application space and market in the fields of controlling soil-borne diseases of crops and promoting plant growth.

### DESCRIPTION OF THE DRAWINGS

FIG. 1 is a diagram showing the colony PCR verification of the Pseudomonas protegens mutant strain Pf5-ΔretS of the present invention.

FIG. 2 is a schematic diagram showing the NiF nitrogen-fixing gene island in the genomic DNA of Pseudomonas stutzeri DSM4166 of the present invention.

FIG. 3 is a diagram showing the enzymatic identification of the expression plasmid pBeloBAC11-oriT-TnpA-genta-NiF constructed by the Red/ET direct cloning method of the present invention by restriction endonuclease Kpn I.

FIG. 4 is a flow chart showing the expression plasmid pBeloBAC11-oriT-TnpA-genta-NiF constructed by the Red/ET direct cloning method of the present invention.

FIG. 5 is a diagram showing the colony PCR verification of the nitrogen-fixing Pseudomonas protegens strain Pf5-NiF of the present invention.

FIG. 6 is a diagram showing the colony PCR verification of the nitrogen-fixing mutant Pseudomonas protegens strain Pf5-ΔretS-NiF of the present invention.

FIG. 7 is a bacteriostatic test of Pseudomonas protegens Pf5 and its mutant strains Pf5-NiF, Pf5-ΔretS and Pf5-ΔretS-NiF of the present invention against Bacillus subtilis.

8A to 8C are schematic diagrams showing the effects of the nitrogen-fixing Pseudomonas syringae Pf5-NiF treatment and the application of the control nitrogen fertilizers NO₃⁻ and Pf5 on Arabidopsis thaliana 4 weeks after transplanted into a pot.

### DEPOSIT INFORMATION

Classification designation: Pseudomonas protegens mutant strain Pf5-NiF

Name of the depository: China General Microbiological Culture Collection Center Institute of Microbiology

Address of the depository: NO. 1 West Beichen Road, Chaoyang District, Beijing 100101, China

Deposit date: Mar. 28, 2017

Deposit number: CGMCC NO. 13948

Classification designation: Pseudomonas protegens mutant strain Pf5-ΔretS

Name of the depository: China General Microbiological Culture Collection Center Institute of Microbiology

Address of the depository: NO. 1 West Beichen Road, Chaoyang District, Beijing 100101, China

Deposit date: Mar. 28, 2017

Deposit number: CGMCC NO. 13949

Classification designation: Pseudomonas protegens mutant strain Pf5-ΔretS-NiF

Name of the depository: China General Microbiological Culture Collection Center Institute of Microbiology

Address of the depository: NO. 1 West Beichen Road, Chaoyang District, Beijing 100101, China

Deposit date: Mar. 28, 2017

Deposit number: CGMCC NO. 13950

### DETAILED DESCRIPTION

The present invention will be further described in conjunction with the drawings and the Examples. The following description is to explain the present invention and will not limit its contents.

### Example 1

A method for screening Pseudomonas protegens Pf5 mutant strain Pf5-ΔretS, comprising the specific steps as follows:

(1) The plasmid pBBR1-Rha-TEGpsy-kan (which can express recombinases in Pseudomonas) was introduced into the wild type Pseudomonas protegens Pf5 by electrotransformation. The electrotransformed bacteria were coated on a plate of LB medium+kanamycin (kin, 30 μg/mL), and 12 single colonies were selected randomly to extract the plasmids to be enzymatically identified, and the correct transformant Pf5::pBBR1-Rha-TEGpsy-kan was screened;

(2) retS gene in the genome of Pseudomonas protegens Pf5 was knocked out. The linear DNA fragment loxM-genta (which was obtained by PCR method using a pair of primers, RetS-Genta-loxM-5′ GCACACGCCCTTGCCGTGCGGTCATTACGCCGCGCATAGTTATAA TCAGGCATCAACCAACGAAGGGATTTCGCCAGCTGAATTACATTC CCAACCG/RetS-Genta-loxM-3′ TGGAGCATGGTGGGAGCTCACGAC TAAAGGAGGGCGAGCGAGAGTTTAACAGGCGCCGCAGAGCCTGT CGGCTCACAACTTAAATGTGAAAGTGGGTC, shown in SEQ ID NO. 15 and SEQ ID NO. 16 respectively) was electrotransformed into Pf5::pBBR1-Rha-TEGpsy-kan obtained in step (1). Using the method of Red/ET homologous recombination, under the action of the recombinase, retS gene in the genome of Pseudomonas protegens Pf5 was replaced by gentamicin resistance gene (genta). Multiple single colonies were selected randomly to be subject to PCR verification (the pair of primers used for verification are check-5′ TGCTTCTACCGCAAGGACATC/check-3′ GCTGATGAAGCAC GAGAGCAC, shown in SEQ ID NO. 13 and SEQ ID NO. 14 respectively). The correct transformant Pf5::ΔretS-genta-loxM was screened.

The genta resistance gene in Pf5::ΔretS-genta-loxM was eliminated. PCM157 plasmid capable of expressing Cre recombinase was electrotransformed into Pf5::ΔretS-genta-loxM, and was coated on a plate of LB medium+tetracycline (tet 25 μg/mL) for screening. The resultant recombinants were inoculated into 1 mL LB+tet 25 μg/mL liquid medium, and cultured at 900 rpm, 30° C. overnight. 50 μL of the overnight cultured bacterial solution was transferred to 1 mL of fresh LB+tet 25 μg/mL liquid medium. After culturing at 900 rpm for 3 hours at 30° C., 1 mM of isopropyl-β-D-thiogalactoside (IPTG) was added for induction. After continuing the culture for 2 hours, the bacterial solution was streaked in a Z-shaped line on a LB plate with a blue inoculation loop. After single colonies grew, they were double-streaked on a LB plate and a LB+genta 15 μg/mL plate respectively and cultured at 30° C. overnight. If single colonies grew on both plates, it indicated that the genta resistance gene in the recombinant had not been eliminated; If the colonies grew on LB plate while did not grow on the LB+genta 15 μg/m plate, it indicated that the genta resistance gene in the recombinant had been eliminated. Such recombinants whose genta resistance gene had been eliminated were picked up and subjected to colony PCR verification and sequencing, using the following primers:

check-5′ TGCTTCTACCGCAAGGACATC/

check-3′ GCTGATGAAGCACGAGAGCAC; as shown in SEQ ID NO. 13 and SEQ ID NO. 14 respectively.

(3) The correct transformant Pf5-ΔretS was cryopreserved after PCR verification and sequencing for subsequent bacteriostatic, room temperature potting and field trials.

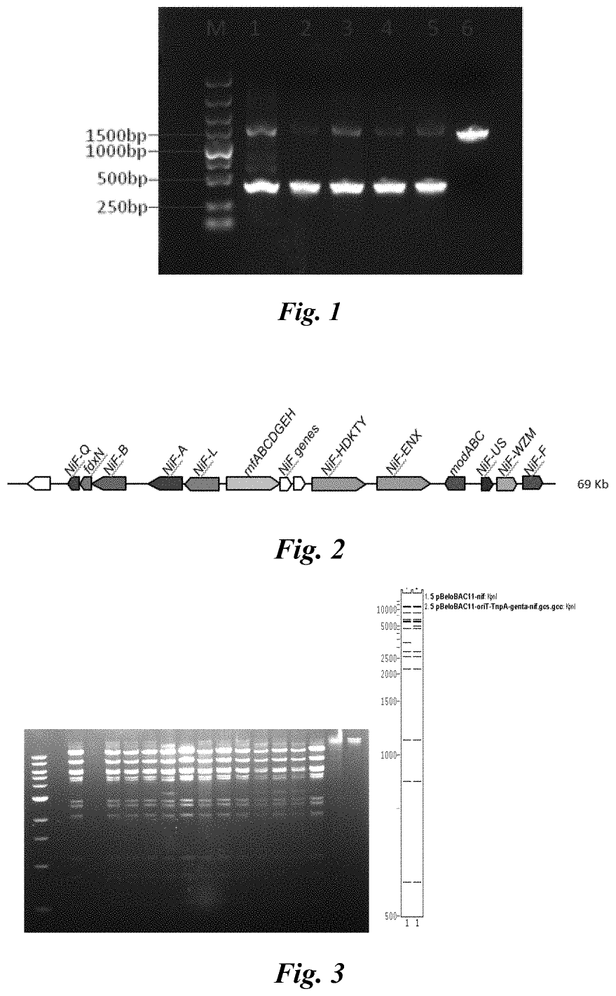

FIG. 1 shows that M is the marker of DL 5,000 DNA, samples 1-5 are the final transformant Pf5-ΔretS, and sample 6 is Pf5::ΔretS-genta-loxM. Under the action of the Cre recombinase introduced by IPTG, specific recombination between two loxM sites (sequences) was mediated, and the genta resistance gene sequence between the loxM sites was deleted. Therefore, the effects of the exogenous resistance gene on the growth, reproduction and colonization of Pseudomonas protegees Pf5 were eliminated. It made the strain safer to be used.

### Example 2

A method for screening Pseudomonas protegens mutant strain Pf5-NiF, comprising the specific steps as follows:

(1) Using Red/ET direct cloning method, the restriction endonucleases Afl II and Ssp I were used to digest the genomic DNA of Pseudomonas stutzeri DSM4166 to obtain a 69 kb NiF nitrogen-fixing gene island (FIG. 2), which was verified by DNA fragment gel electrophoresis, and ligated to the corresponding vector. The primers used were:

Primer 1: AGTGAATTGTAATACGACTCACTATAGGGCGAATT CGAGCTCGGTACCCGCTTAAGTACGGCTACCTGGAGCTCGCGCCA GTG, as shown in SEQ ID NO.

Primer 2: TACGGCTACCTGGAGCTCGCGCCAGTGCTTGCCGAC ATCGAATCACGGCCGCTGCTGCAGCACGTGGTGGTCACCGGCCG GGATCCGTTTAAACACAAATGGCAAGGGCTAATG, as shown in SEQ ID NO. 2;

Primer 3: ATTGATGTTTTCCTTGGCCAGCGCCTCGAACATCCG GCTGGCGACGCCTGCGTGCGAACGCATACCGACACCGACGATAG GGATCCGTTTAAACGGTGTGGTAGCTCGCGTATT, as shown in SEQ ID NO. 3;

Primer 4: GCGACACTATAGAATACTCAAGCTTGGCATGAAT GCAGGTCGACTCTAGAGAATATTGATGTTTTCCTTGGCCAGCGCC TCGAAC, as shown in SEQ ID NO. 4.

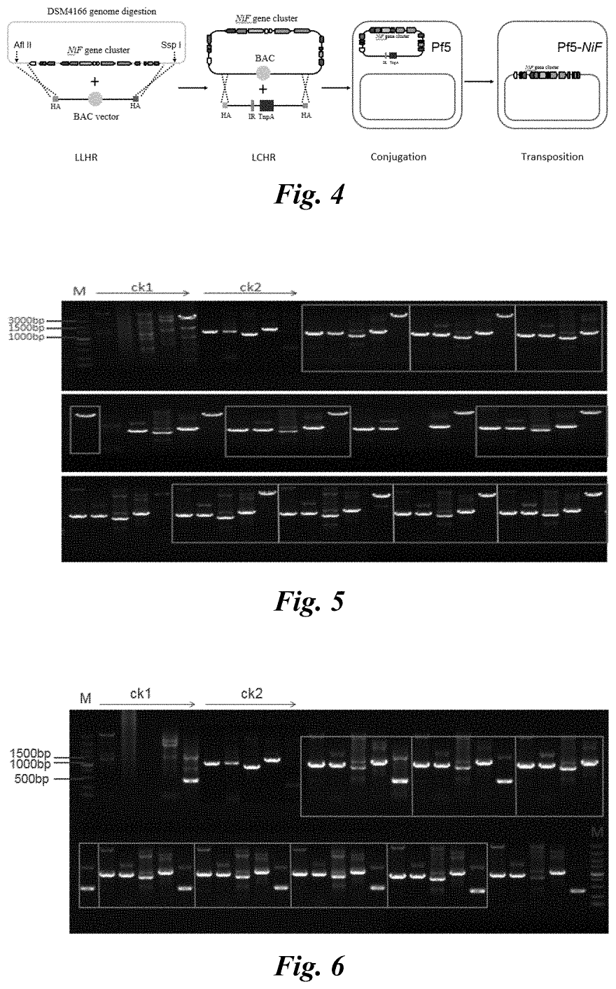

The expression plasmid pBeloBAC11-oriT-TnpA-genta-NiF (FIG. 3) was constructed and was identified by digesting with restriction endonuclease Kpn I. The correct plasmid was electrotransformed into E. coli ET12567 (FIG. 4).

(2) The plasmid pBeloBAC11-oriT-TnpA-genta-NiF from E. coli ET12567 was introduced into Pseudomonas protegens Pf5 by conjugative transfer, and then NiF gene was randomly inserted into the genomic DNA of Pf5 by transposition. The detailed operation of the conjugation transfer was as follows: A single colony of Pseudomonas protegens Pf5 was picked up and was cultured (LB medium, 30° C.) separately with E. coli ET12567 (LB+genta 2 μg/mL+cm 10 μg/mL+kin 1 μg/mL medium, 37° C.) overnight; The two overnight bacterial solutions were centrifuged at 7000 rpm for 1 minute. Pseudomonas protegens Pf5 and E. coli ET12567 were washed twice with fresh LB medium, resuspended in 300 μL of LB medium. 50 μL of each suspension was mixed and coated on a small area in the middle of the LB plate and air dried. After incubating for 4 hours at 37° C., the plate was invertedly incubated in an incubator at 30° C. overnight; The bacteria on the plate were scraped with an inoculating loop, mixed thoroughly with 1 mL sterilized solution. 100 μL of bacterial solution was streaked in a Z-shaped line on a plate of PMM medium+genta 25 μg/mL, and cultured invertedly at 30° C. for 2 days until single colonies appeared; Two days later, colonies grew. A single colony was picked up to be inoculated in 1 mL of LB+genta 25 μg/mL and to be cultured overnight, followed by colony PCR verification using the following 5 pairs of primers:

NiF-check-1 GGTCTACCAGCTCGACCT/

NiF-check-2 CGATTCCAGCGTCGAATGAT;

NiF-check-3 GCTGACCTCCTTGAGGTGCT/

NiF-check-4 CAGCGGCACCTCGAGGAGT;

NiF-check-5 GATAGAGCAGGTCCTCGAT/

NiF-check-6 GGTGCTCTACGTCAGCCATT;

NiF-check-7 CGACAGATCCTGATTACCGT/

NiF-check-8 TACCCTCGACCAGCTTGAGCA;

check-5′ TGCTTCTACCGCAAGGACATC/

check-3′ GCTGATGAAGCACGAGAGCAC; as shown in SEQ ID NO. 5 to SEQ ID NO. 14, respectively.

The first four pairs of primers were used to verify whether the NiF nitrogen-fixing gene had been integrated as a whole into the genome of Pseudomonas protegens Pf5. The amplified PCR fragments were 1000 bp, 970 bp, 830 bp, and 1080 bp, respectively. The fifth pair of primers was used to verify that the strain to which the NiF nitrogen-fixing gene was introduced was Pseudomonas protegens Pf5 instead of Escherichia coli ET12567, and the PCR amplification result was retS gene with a DNA fragment size of 3200 bp.

The correct transformant Pf5-NiF was sent to the sequencing after colony PCR verification, and that with the correct results was cryopreserved and used for subsequent bacteriostatic, room temperature potting and field trials.

FIG. 5 shows that M is the marker of DL 5,000 DNA, ck1 is wild type Pseudomonas protegens Pf5, and ck2 is Escherichia coli ET12567, which two serve as control groups. 5 columns of DNA electrophoresis maps represent that one Pf5-NiF transformant was subjected to colony PCR verification with the above 5 pairs of primers. After repeated careful comparison, the correct transformant Pf5-NiF was marked with a blue box. Thus, it was proved that the NiF nitrogen-fixing gene in Pseudomonas stutzeri DSM4166 had been integrated into the genome of Pseudomonas protegens Pf5 as a whole, and the correct transformant Pf5-NiF was obtained.

### Example 3

A method for screening Pseudomonas protegens mutant strain Pf5-ΔretS-NiF, comprising the specific steps as follows:

The plasmid pBeloBAC11-oriT-TnpA-genta-NiF from E. coli ET12567 was introduced into mutant Pseudomonas protegens Pf5-ΔretS by conjugative transfer, and then NiF gene was randomly inserted into the genomic DNA of Pf5-ΔretS by transposition. The detailed operation of the conjugation transfer was as follows: The mutant Pseudomonas protegens Pf5-ΔretS (LB medium, 30° C.) and E. coli ET12567 (LB+genta 2 μg/mL medium, 37° C.) were cultured overnight respectively; The next day, the mutant Pseudomonas protegens Pf5-ΔretS and E. coli ET12567 were washed twice with fresh LB medium respectively, and dissolved in 500 μL of LB in the same amounts respectively, and then mixed together to 1 mL. After centrifuged at 9000 rpm for 1 minute, most of the supernatant was discarded. 100 μL of the bacterial solution was retained to be resuspended with the mixed bacteria, and uniformly coated on a LB plate in a small range. After incubated at 37° C. for 4 hours, the plate was placed in an incubator at 30° C. to be cultured overnight; On the third day, the co-cultured bacteria were transferred with a yellow inoculating loop from the LB plate to 1 mL of LB to be mixed thoroughly. 30 μL of the bacterial solution was streaked in a Z-shaped line on PMM medium+genta 25 μg/mL. Two days later, colonies were grown. Single colonies were picked up and inoculated in 1 mL LB+genta 25 μg/mL to be cultured overnight, and then subjected to colony PCR verification using the 5 pairs of primers shown in Example 2. The first four pairs of primers were used to verify whether the NiF nitrogen-fixing gene had been integrated as a whole into the genome of Pseudomonas protegens Pf5. The amplified PCR fragments were 1000 bp, 970 bp, 830 bp, and 1080 bp, respectively. The fifth pair of primers was used to verify that the strain to which the NiF nitrogen-fixing gene was introduced was Pseudomonas protegens Pf5 instead of Escherichia coli ET12567. Because this time the NiF nitrogen-fixing gene was transferred into the mutant Pseudomonas syringae Pf5-ΔretS whose retS gene had been knocked out, the PCR amplification result was a DNA fragment of 400 bp in size. The correct transformant Pf5-ΔretS-NiF was sent to the sequencing after PCR verification, and that with the correct results was cryopreserved and used for subsequent bacteriostatic, room temperature potting and field trials

FIG. 6 shows that M is the marker of 1 kb DNA, ck1 is mutant Pseudomonas protegens Pf5-ΔretS whose retS gene had been knocked out, ck2 is Escherichia coli ET12567, which two serve as control groups. 5 columns of DNA electrophoresis maps represent that one Pf5-NiF transformant was subjected to colony PCR verification with the above 5 pairs of primers. After repeated careful comparison, the correct transformant Pf5-NiF was marked with a blue box. Thus, it was proved that the NiF nitrogen-fixing gene in Pseudomonas stutzeri DSM4166 had been integrated as a whole into the genome of mutant Pseudomonas protegens Pf5-ΔretS whose retS gene had been knocked out, and the correct transformant Pf5-ΔretS-NiF was obtained.

The information about Pseudomonas protegees Pf5 and its mutant strains Pf5-NiF, Pf5-ΔretS and Pf5-ΔretS-NiF obtained by the present invention is shown in Table 1.

TABLE 1
Information about each strain

| Strains of | | |
|---|---|---|
| Pseudomonas | | |
| protegens | Relevant properties | origins |
| Pf5 | Wild type | German Collection |
| | | of Microorganisms, |
| | | DSMZ |
| Pf5-NiF | Pf5 genetically engineered strain having integrated NiF | The present |
| | nitrogen-fixing gene, having genta resistance during | invention |
| | culture, and capable of biologically nitrogen-fixing and | |
| | reducing the usage of nitrogen fertilizer during the | |
| | growth of plants | |
| Pf5-ΔretS | Pf5 mutant strain whose retS gene has been knocked | The present |
| | out, having no genta resistance gene, having no | invention |
| | resistance during culture, and having increased bacteria | |
| | killing activity | |
| Pf5-ΔretS-NiF | Pf5 genetically engineered strain whose retS gene has | The present |
| | been knocked out and having integrated NiF | invention |
| | nitrogen-fixing gene, having genta resistance during | |
| | culture, having increased bacteria killing activity, and | |
| | capable of biologically nitrogen-fixing and reducing the | |
| | usage of nitrogen fertilizer during the growth of plants | |

### Example 4

The filter paper method was used to detect the inhibitory effects of Pseudomonas protegens Pf5 and its mutant strains Pf5-NiF, Pf5-ΔretS and Pf5-ΔretS-NiF on Bacillus subtilis. The specific steps were as follows:

(1) Bacillus subtilis and the experimental strain Pseudomonas protegens Pf5 and its mutant strains Pf5-NiF, Pf5-ΔretS and Pf5-ΔretS-NiF were inoculated into 1 mL LB liquid medium respectively, and were cultured at 900 rpm, overnight at 30° C.;

(2) The next day, Bacillus subtilis was centrifuged at 9000 rpm for 1 minute, and 100 μL of the bacterial solution was uniformly coated on a LB solid plate. After dried, several double-layer filter paper sheets having a diameter of 6 mm were placed on the plate. 5 μL overnight cultured experimental strain Pseudomonas protegens Pf5 and its mutant strains Pf5-NiF, Pf5-ΔretS and Pf5-ΔretS-NiF were added dropwise to the filter paper sheets. The plate was cultured at 30° C. overnight.

(3) On the third day, the size of the inhibition zone around each small filter paper sheet on the plate was observed.

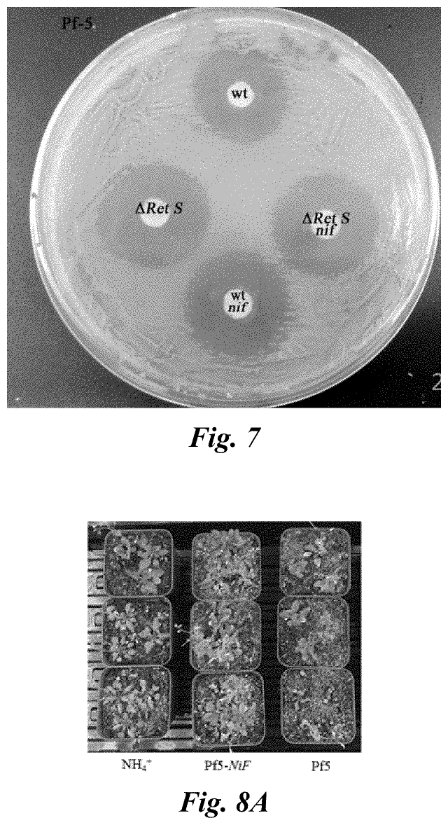

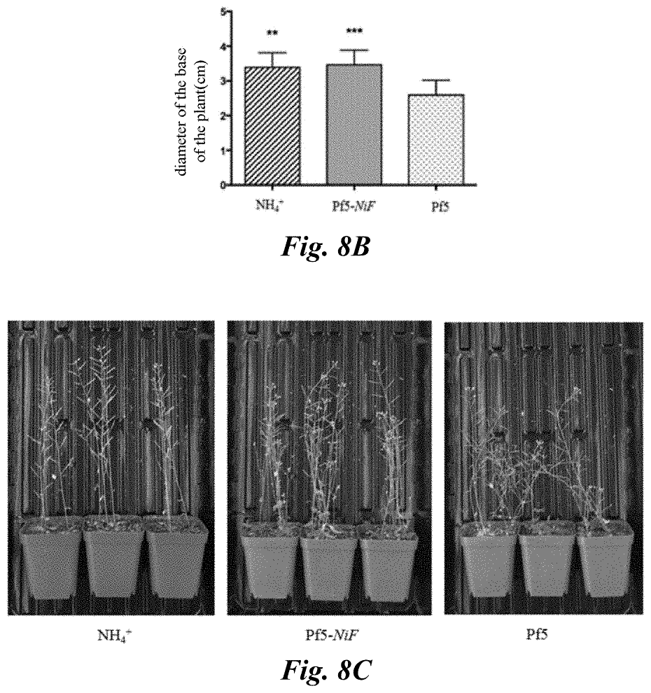

FIG. 7 shows that the inhibition zones of the Pseudomonas protegens Pf5 mutant strains Pf5-ΔretS and Pf5-ΔretS-NiF whose retS gene had been knocked out were much larger than those of Pseudomonas protegens Pf5 and Pf5-NiF whose retS gene had not been knocked out. It indicated that the ability for inhibiting Bacillus subtilis was increased after ΔretS gene was knocked out.

### Example 5

The room temperature pot experiment of Pseudomonas protegens strain Pf5-NiF was carried out with Arabidopsis thaliana as the test subject. The test protocol was as follows:

1) Wild type Arabidopsis thaliana Col-0 was used as the test subject. The test conditions were: temperature 20° C., light intensity 80 μmol·m⁻²·s⁻¹, light cycle: 16 hours light, 8 hours dark; the test was divided into 3 groups:

i) Normal application of nitrogen fertilizer 1 mM (NO₃⁻), as the control

ii) No nitrogen fertilizer was applied, but the normal Pseudomonas protegens strain Pf5 was applied.

iii) No nitrogen fertilizer was applied, but the nitrogen-fixing Pseudomonas protegees strain Pf5-NiF was applied.

2) pre-treatment of the seeds of Arabidopsis thaliana: Seeds were placed in a refrigerator at 4° C. for 2-4 days (the seeds were vernalized to keep the germination rate of the batch of seeds tested consistent); Disinfection of the seeds: After detoxification in 2 w.t. % sodium hypochlorite (NaClO) (continuous shaking during detoxification to allow the seeds to be fully contacted) for 15 minutes, the seeds were then rinsed 5-10 times with sterile water;

3) The seeds of Arabidopsis thaliana were sown on ½ MS solid medium (the seeds at the same location should not be excessive). Specific operation: the supernatant in the Ep tube was sucked up with a pipette tip. 200 μL of the medium was aspirated, and the liquid was slowly flowed to the tip of the pipette, and then gently spotted on the MS medium. The plate was then incubated vertically.

4) Transplant of the seedlings: When the seedlings grew on the MS medium (took about 10 days), they were transplanted into the soil medium (roseite: black soil (mass ratio)=1:1), three seedlings per pot. The roots of the seedlings were not broke during the transplant process.

5) Inoculation: Pf5-NiF single colonies on the plate were cultured overnight on KB medium, and inoculated in LB medium at a ratio of overnight bacterial solution:fresh medium (volume ratio)=1:50 on the next day, and cultured to 220 rpm, 30° C. until OD₆₀₀=0.6 (the number of Pf5-NiF can reach 1×10⁹ cfu/mL). 1 mL of bacterial solution was inoculated in the range of 0.5 mm around the rhizosphere of the seedlings of Arabidopsis thaliana for 3 days.

6) Cultivation in the pots in the greenhouse according to the set test conditions,

The composition of each medium used in the potting test at room temperature was as follows:

MS medium: NH₄NO₃ 1.65 g/L, KNO₃ 1.9 g/L, CaCl₂.2H₂O 0.44 g/L, MgSO₄.7H₂O 0.37 g/L, KH₂PO₄ 0.17 g/L, KI 0.83 mg/L, H₃BO₃ 6.2 mg/L, MnSO₄.4H₂O 22.3 mg/L, ZnSO₄.7H₂O 8.6 mg/L, Na₂MoO₄.2H₂O 0.25 mg/L, CuSO₄.5H₂O 0.025 mg/L, CoCl₂.6H₂O 0.025 mg/L, FeSO₄.7H₂O 27.8 mg/L, Na₂-EDTA.2H₂O 37.3 mg/L, inositol 100 mg/L, nicotinic acid acid 0.5 mg/L, vitamin B₆ 0.5 mg/L, vitamin B₁ 0.1 mg/L, glycine 2 mg/L.

KB medium: K₂HPO₄ 0.1 g/L, KH₂PO₄ 0.4 g/L, NaCl 0.1 g/L, MgSO₄.7H₂O 0.01 g/L, Fee (SO₄)₃.H₂O 0.01 g/L, ZnSO₄.7H₂O 0.01 g/L, MnCl₂H₂O 0.01 g/L, NaMoO₄ 0.01 g/L, CaCl₂ 2H₂O 0.1 g/L, sodium citrate 1 g/L, glucose 5.5 g/L, yeast extract 0.2 g/L, pH adjusted to 7.0.

FIG. 8A to FIG. 8C show that after 4 weeks of pot experiment at room temperature, in group ii) to which no nitrogen fertilizer was applied but normal Pseudomonas protegens strain Pf5 was applied, the growth of Arabidopsis thaliana was not good, the leafs were small and the stems were short, and was much worse than other two experiment groups (FIGS. 8A and 8C). While in group iii) to which no nitrogen fertilizer was applied but Pseudomonas protegens strain Pf5-NiF was applied, the phenotypes such as growth of Arabidopsis thaliana and the size of the leafs was even better than group i) to which nitrogen fertilizer was applied (FIG. 8C). It was because Pf5-NiF can be used for biological nitrogen fixation and thus reduced the usage of nitrogen fertilizer. Moreover, due to its own bactericidal and plant growth-promoting effects, the Pseudomonas protegens strain Pf5 enabled plants to thrive and exceeded the control group in various aspects (FIGS. 8A and 8C).

The green quality of potted Arabidopsis thaliana treated with the Pseudomonas protegens strain Pf5-NiF obtained by the present invention was increased by about 8% on average (FIG. 8B), and the plants appeared greener and more robust without applying nitrogen fertilizer.

### Example 6

Effects of Pf5 Engineered Strain on Wheat Growth and Soil-Borne Disease Control

1. Test time: October 2016-May 2017

2. Test location: Xinzhai Town, Yucheng City, Dezhou City, Shandong Province

3. Test crop: wheat

4. Test treatment:

During the wheat planting period, the fertilizer management and the addition of microbial agents were according to the conventional method, that is, the base fertilizer was equivalent to pure N 225 kg/hm², P₂O₅ 180 kg/hm², K₂O 180 kg/hm², and pure N 80 kg/hm² was applied during the shooting period. Frozen water (December 4th) and shooting water (April 10th) was normally irrigated during the whole growth period at the amount of 750 m³/hm². The dosage form of the microbial agents is liquid, and the effective viable bacterial count was ≥5 billion/ml. The application amount was 2 kg/mu, and the application method was seed dressing and root irrigation.

Treatment 1: blank control (CK) without applying any microbial agent

Treatment 2: application of the control microbial agent, which was a commercially available conventional Pseudomonas protegees Pf5 agent (purchased from Lanling Pharmaceutical Co., Ltd., Changzhou, Jiangsu);

Treatment 3: application of the test microbial agent having the active ingredient Pf5-NiF;

Treatment 4: application of the test microbial agent having the active ingredient Pf5-ΔretS;

Treatment 5: application of the test microbial agent having the active ingredient Pf5-ΔretS-NiF;

Treatment 6: application of the test microbial agent having the active ingredient Pf5-ΔretS-NiF; wherein the application amount of nitrogen fertilizer was ⅔ of the standard fertilization, and the phosphorus and potassium were consistent.

The field plot test results are shown in Tables 2 and 3.

TABLE 2
Prevention effects of different treatments in field plots on take-all disease of wheat

| | | | | 120 d after sowing | | |
|---|---|---|---|---|---|---|
| | Late | | Height of the | Ration of | Index of | |
| | emergence | Emergence | seedlings 30 d | diseased | the | Control |
| | time | rate | after sowing | plants | disease of | effects |
| treatments | (d) | (%) | (cm) | (%) | the root | (%) |
| Treatment 1 | 2 | 94 | 30.6 | 96 | 78.2 | — |
| Treatment 2 | 0 | 99 | 33.5 | 46 | 24.2 | 52.1 |
| Treatment 3 | 0 | 100 | 34.0 | 44 | 23.9 | 54.3 |
| Treatment 4 | 0 | 100 | 41.9 | 18 | 13.4 | 77.8 |
| Treatment 5 | 0 | 100 | 42.6 | 16 | 12.7 | 79.1 |
| Treatment 6 | 0 | 100 | 42.3 | 17 | 13.2 | 78.2 |

As can be seen from the results in Table 2, the three Pf5 engineered bacteria (treatments 4, 5, 6), whose retS gene had been knocked out, had 77.8%, 79.1%, and 78.2% control effects on take-all disease of wheat. The control effects of the three were all significantly higher than that of the two Pf5 bacteria (treatment 2, 3) whose retS gene had not been knocked out (52.1% and 54.3%). After the application of Pf5 microbial agent, there was effect on the height of the seedlings of the wheat within 30d after sowing. Moreover, the effects of applying Pf5 engineered bacteria whose retS gene had been knocked out were more significant, which were generally 20% higher than that of the non-knocked out Pf5 treatment group. Moreover, due to the presence of nitrogen-fixing gene, after the application of nitrogen fertilizer decreased by ⅓, treatment 6 still maintained the effect same as that without the decrease of the application, indicating that the nitrogen-fixing gene played an important role in the growth of wheat.

TABLE 3
Effects of different treatments in field plots
on the yield and constitutive factors of wheat

| | Constitution of yield | | | | |
|---|---|---|---|---|---|
| | | | Weight of | | The increase |
| | Number of | Number of ears | thousand grains | yield | of yield |
| treatments | ear kernels | (ten thousand/hm²) | (g) | (kg/hm²) | (%) |
| Treatment 1 | 26.8 | 490 | 25.6 | 6142.2 | — |
| Treatment 2 | 34.1 | 530 | 33.1 | 6744.1 | 9.79 |
| Treatment 3 | 34.5 | 535 | 33.3 | 6742.8 | 9.83 |
| Treatment 4 | 41.6 | 665 | 40.7 | 7780.2 | 26.67 |
| Treatment 5 | 42.3 | 667 | 41.2 | 7826.1 | 27.42 |
| Treatment 6 | 42.2 | 665 | 40.9 | 7810.3 | 27.16 |

As shown in table 3, after applying the Pf5 microbial agent, the number of the ears of wheat was significantly increased. The three Pf5 engineered bacteria (treatments 4, 5, 6), whose retS gene had been knocked out, had larger increase, generally around 35%. In the treatment group to which Pf5 microbial agent was applied, the yield of wheat was also significantly higher than that of the control (treatment 1), which increased by about 26%. Under the action of nitrogen-fixing gene NiF, treatment 6 which had ⅓ decrease of nitrogen fertilizer, also obviously increased the yield of wheat, indicating that it was feasible to apply nitrogen-fixing engineered bacteria to reduce the application of nitrogen fertilizer during the growth of wheat.

### Example 7

Report of Field Test on Garlic with Pf5 Engineered Strains

1. Test time: October 2016-June 2017

2. Test location: Qianjiang Village, Yucheng Town, Yutai County, Jining City, Shandong Province

3. Test crop: hybrid garlic (white garlic)

4. Test treatments: The test was divided into 6 treatments as follows:

Treatment 1: Fertilizer was applied according to the farmers' convention (N 45 kg/hm², P₂O₅ 22.5 kg/hm², K₂O 22.5 kg/hm², organic fertilizer 40 kg/mu, high-nitrogen and high-potassium compound fertilizer for topdressing);

Treatment 2: Optimized fertilization, N 30 kg/hm², P₂O₅ 16 kg/hm², K₂O 24 kg/hm², N 30 kg/hm², P₂O₅ 16 kg/hm², K₂O 24 kg/hm², bio-organic fertilizer 200 kg/hm², formula fertilizer used for topdressing (18-5-17 humic acid type) 20 kg/mu; Dodine was used for seed dressing; hymexazol, methyl thiophanate and dodine were applied according to the actual situation in the spring when topdressing;

Chemical control measures (according to actual re-selection): mepiquat and brassinolide

Treatment 3: Microbial agent—Pseudomonas protegens Pf5-NiF

Fertilization was consistent with optimized fertilization. Pseudomonas protegens was used for seed dressing. Solution of Pseudomonas protegens was flushed by water to be applied in the spring. Bacterial solution was flushed by water to be applied during topdressing.

Treatment 4: Microbial agent—Pseudomonas protegens Pf5-ΔretS

Fertilization was consistent with optimized fertilization. Pseudomonas protegens was used for seed dressing. Solution of Pseudomonas protegens was flushed by water to be applied in the spring. Bacterial solution was flushed by water to be applied during topdressing.

Treatment 5: Microbial agent—Pf5-ΔretS-NiF

Fertilization was consistent with optimized fertilization. Pseudomonas protegens was used for seed dressing. Solution of Pseudomonas protegens was flushed by water to be applied in the spring. Bacterial solution was flushed by water to be applied during topdressing.

Treatment 6: Microbial agent—Pf5-ΔretS-NiF

The application amount of nitrogen fertilizer was ⅔ of optimized fertilization, and phosphorus and potassium were same as optimized fertilization. Others were consistent with optimized fertilization. Pseudomonas protegens was used for seed dressing. Solution of Pseudomonas protegens was flushed by water to be applied in the spring. Bacterial solution was flushed by water to be applied during topdressing.

The dosage form of the microbial agents was liquid, and the effective viable bacterial count was ≥5 billion/ml. The application amount was 2 kg/mu.

On Jan. 26, 2017, the length and width of the garlic leaves, the diameter of the stein, and the enzymatic activity of the root were measured before wintering (Table 4).

TABLE 4
Statistical table of biological traits of the test treatments

| | | | | | | Enzymatic |
|---|---|---|---|---|---|---|
| | | | | | | activity of root |
| | | Diameter | | Weight of | Weight of | mg/g |
| | Length of | of stem | Width of | seedling | root | (fresh weight of |
| treatment | leaf (cm) | (cm) | leaf | (g) | (g) | root)/h |
| Treatment 1 | 21.4 | 11.82 | 1.008 | 105.33 | 14.21 | 0.367 |
| Treatment 2 | 22.3 | 12.62 | 0.992 | 131.12 | 18.75 | 0.384 |
| Treatment 3 | 23.1 | 12.89 | 1.047 | 134.46 | 19.68 | 0.409 |
| Treatment 4 | 24.8 | 13.75 | 1.179 | 139.81 | 21.05 | 0.422 |
| Treatment 5 | 25.5 | 14.58 | 1.124 | 144.73 | 21.96 | 0.436 |
| Treatment 6 | 25.1 | 14.10 | 1.121 | 143.65 | 21.32 | 0.423 |

The measured data were consistent with the results of field test observations. Four treatments of Pseudomonas protegens showed that the leaves were significantly longer and wider in the early growth stage of the garlic. The leaf lengths of treatments 5 and 6 were increased by 19.16 and 17.29%, respectively, and the leaf lengths of treatments 5 and 6 observed in the field were more prolonged than the control treatment. By measuring the enzymatic activity of the root of the garlic before wintering, the enzymatic activity of the root of the four treatments using Pseudomonas protegens was significantly higher than that of the control treatment. Donine inhibited the growth of pathogens and also harmed the beneficial bacteria around the garlic so that indirectly hindered the growth of garlic in the early stage of growth. The Pseudomonas protegens agents replaced the seed dressing with donine. Therefore, the Pseudomonas protegens treated garlic seedlings grew vigorously.

From the planting of garlic in October to the harvest in May of the following year, the production was calculated and converted into the yield of garlic, and the results were statistically analyzed.

The garlic yield results of test treatments 1-6 are shown in Table 5.

TABLE 5
Effects of different treatments on garlic yield and garlic quality

| | | Increase in | Vc in the | | Soluble | |
|---|---|---|---|---|---|---|
| | yield | yield | garlic | Soluble sugar | protein | Allicin |
| treatment | (kg/667 m²) | (%) | (%) | (mg//Gfw) | (mg/gFW) | (μg/gFW) |
| Treatment 1 | 18345 | — | 16 | 20 | 27 | 20 |
| Treatment 2 | 24130 | 31.5 | 18 | 27 | 44 | 26 |
| Treatment 3 | 25084 | 36.7 | 19 | 29 | 47 | 29 |
| Treatment 4 | 29268 | 59.6 | 23 | 38 | 67 | 47 |
| Treatment 5 | 29846 | 62.7 | 25 | 43 | 74 | 49 |
| Treatment 6 | 29653 | 61.6 | 24 | 41 | 72 | 47 |

It can be seen from Table 5, the four treatments of Pseudomonas protegens had a very positive effect on the yield increase of garlic. Treatments 5 and 6 were increased by 62.7% and 61.6%, respectively, compared with the control treatment, indicating that the effect of Pf5 microbial agents was very obvious in the case of reduced nitrogen fertilizer application. For the determination of garlic quality, there were four indicators: Vc in the garlic, soluble sugar content, soluble protein and allicin. Each of the four treatments of Pseudomonas protegens had a very significant improvement. After reducing the application of nitrogen fertilizer, compared with the control treatment, the content of Vc in the garlic of treatments 5 and 6 were increased by 25% and 24%, the soluble sugar content increased by 115% and 105%, the soluble protein content increased by 174% and 167%, and the dry content of allicin increased by 145% and 135%. It can be seen that the application of Pf5 microbial agents had a great impact on the quality of garlic. It can bring huge economic benefits to customers, and reduce ⅓ of the usage of nitrogen fertilizer. Under the premise of not affecting the quality of the product, the production cost of the customer can be reduced, and the effect is immediate.

The above description of the specific embodiments of the present invention has been described with reference to the accompanying drawings, but is not intended to limit the scope of the present invention. On the basis of the technical solutions of the present invention, various modifications or variations that can be made by the skilled in the art without any creative work are still within the scope of protection of the present invention.

1. Pseudomonas protegens Pf5 mutant strain Pf5-NiF, Pf5-ΔretS or Pf5-ΔretS-NiF, having the deposit numbers CGMCC NO. 13948, CGMCC NO. 13949 and CGMCC NO. 13950 respectively.

2. (canceled)

3. (canceled)

4. A composition, for example, a microbial agent, which has any one of Pf5-NiF, Pf5-ΔretS or Pf5-ΔretS-NiF according to claim 1, or any combination thereof as the active ingredient.

5. A method for producing Pseudomonas protegens Pf5 mutant strain Pf5-NiF, comprising cloning the whole NiF nitrogen-fixing gene island in the genome of Pseudomonas stutzeri DSM4166 into the genome of Pseudomonas protegens Pf5, and expressing the NiF nitrogen-fixing gene island to obtain the genetically engineered strain Pf5-NiF.

6. The method according to claim 5, characterized in the following steps:
(1) Using Red/ET direct cloning method, using the restriction endonucleases Afl II and Ssp I to digest the genomic DNA of Pseudomonas stutzeri DSM4166 to obtain a 69 kb NiF nitrogen-fixing gene island, which is ligated to the corresponding vector after verified to be correct by DNA fragment gel electrophoresis; constructing the expression plasmid pBeloBAC11-oriT-TnpA-genta-NiF using the primers as shown in SEQ ID NO. 1, SEQ ID NO. 2, SEQ ID NO. 3 and SEQ ID NO. 4; identifying by digesting with restriction endonuclease Kpn I; electrotransforming the correct plasmid into E. coli ET12567;
(2) Introducing the plasmid pBeloBAC11-oriT-TnpA-genta-NiF from E. coli ET12567 into Pseudomonas protegens Pf5 by conjugative transfer; and then randomly inserting NiF gene into the genomic DNA of Pf5 by transposition;
(3) Sequencing the correct transformant Pf5-NiF after colony PCR verification, and cryopreserving that with the correct results.

7. A method for producing Pseudomonas protegens Pf5 mutant strain Pf5-ΔretS, comprising scarlessly knocking out retS gene from the genome of Pseudomonas protegens Pf5 to obtain the genetically engineered strain Pf5-ΔretS.

8. The method according to claim 7, characterized in the following steps:
(1) Introducing the plasmid pBBR1-Rha-TEGpsy-kan into the wild type Pseudomonas protegens Pf5 by electrotransformation, and screening the correct transformant Pf5::pBBR1-Rha-TEGpsy-kan;
(2) Knocking out retS gene in the genome of Pseudomonas protegens Pf5;
(3) Cryopreserving the correct transformant Pf5-ΔretS after PCR verification and sequencing.

9. A method for producing Pseudomonas protegens Pf5 mutant strain Pf5-ΔretS-NiF, comprising introducing NiF into mutant Pseudomonas protegens Pf5-ΔretS, and then randomly inserting NiF into the genomic DNA of Pf5-ΔretS by transposition.

10. The method according to claim 9, characterized in the following steps:
(1) Introducing the plasmid pBBR1-Rha-TEGpsy-kan into the wild type Pseudomonas protegens Pf5 by electrotransformation, and screening the correct transformant Pf5::pBBR1-Rha-TEGpsy-kan;
(2) Knocking out retS gene in the genome of Pseudomonas protegens Pf5 to obtain the mutant Pseudomonas protegens Pf5-ΔretS;
(3) Using Red/ET direct cloning method, using the restriction endonucleases Afl II and Ssp I to digest the genomic DNA of Pseudomonas stutzeri DSM4166 to obtain a 69 kb NiF nitrogen-fixing gene island, which is ligated to the corresponding vector after verified to be correct by DNA fragment gel electrophoresis; constructing the expression plasmid pBeloBAC11-oriT-TnpA-genta-NiF using the primers as shown in SEQ ID NO. 1, SEQ ID NO. 2, SEQ ID NO. 3 and SEQ ID NO. 4; identifying by digesting with restriction endonuclease Kpn I; electrotransforming the correct plasmid into E. coli ET12567;
(4) Introducing the plasmid pBeloBAC11-oriT-TnpA-genta-NiF from E. coli ET12567 into the mutant Pseudomonas protegens Pf5-ΔretS by conjugative transfer; and then randomly inserting NiF gene into the genomic DNA of Pf5 by transposition.

11. A method for promoting plant growth, killing bacteria and/or fixing nitrogen, comprising administering to a plant or a seed thereof the Pseudomonas protegens mutant strain Pf5-NiF or Pf5-ΔretS-NiF according to claim 1 or a combination thereof, or a composition, for example, a microbial agent, comprising Pseudomonas protegens mutant strain Pf5-NiF or Pf5-ΔretS-NiF according to claim 1 or a combination thereof.

12. A method for promoting plant growth and/or killing bacteria, comprising administering to a plant or a seed thereof the Pseudomonas protegens mutant strain Pf5-ΔretS according to claim 1, or a composition, for example, a microbial agent, comprising the Pseudomonas protegens mutant strain Pf5-ΔretS according to claim 1.
